(19) United States
(12) Patent Application Publication
(10) Pub. No.: US 2004/0146856 A1
(43) Pub. Date: Jul. 29, 2004

(54) Anti-neoplastic viral agents

(76) Inventors: Richard Derek Iggo, Lausanne (CH); Christophe Fuerer, Renens (CH)

Correspondence Address: NIXON & VANDERHYE, PC, 1100 N GLEBE ROAD, 8TH FLOOR, ARLINGTON, VA, 22201-4714, US

(21) Appl. No.: 10/433,681

(22) PCT Filed: Jul. 12, 2002
(86) PCT No.: PCT/GB02/03211

(30) Foreign Application Priority Data
Jul. 13, 2001 (GB) 0117198.2

(51) Int. Cl. C12Q 1/70; C12N 7/00; C12N 15/86

(52) U.S. Cl. 435/5; 435/456; 435/235.1

(57) ABSTRACT

A viral DNA construct, and virus encoded thereby, is provided having one or more tumor specific transcription factor binding sites in place of one or more wild type transcription factor binding sites operatively positioned in the promoter region which controls expression of E1A open reading frame.

Preferred constructs place the tumor specific transcription factor binding sites in operative relation to DNA polymerase, DNA terminal protein and/or DNA binding protein.

Compositions and constructs contained therein are provided, particularly for use in therapy. Methods of treating patients for neoplasms are also provided.

The present invention provides viral agents that have application in the treatment of neoplasms such as tumors, particularly tumors derived from colon cells, more particularly liver tumors that are metastases of colon cell primary tumors. Still more particularly are provided replication competent, and particularly replication efficient, adenovirus constructs that selectively replicate in response to transcription activators present in tumor cells, these factors being present either exclusively or at elevated levels in tumor cells as compared to other cells, and thus which lead to tumor cell death and cell lysis.

By injecting the viral agents of the invention locally into the liver it is possible to treat liver metastases, which are a major cause of morbidity in colon cancer patients. Applications beyond this, e.g. to other sites and other tumors, such as colorectal cancers and melanomas, are also provided.

Viruses which replicate selectively in tumor cells have great potential for gene therapy for cancer as they can spread progressively through a tumor until all of its cells are destroyed. This overcomes the need to infect all tumor cells at the time the virus is injected, which is a major limitation to conventional replacement gene therapy, because in principle virus goes on being produced, lysing cells on release of new virus, until no tumor cells remain. An important fundamental distinction in cancer gene therapy is thus between single hit approaches, using non-replicating viruses, and multiple hit approaches, using replicating viruses.

In practice, only a few cycles of reinfection with the virus can occur before the immune system halts the infection. Even a single cycle of infection should lead to a massive local increase in virus concentration within the tumor, making it possible to achieve the same level of infection of tumor cells after injecting much smaller amounts of replicating than non-replicating viruses. Since the toxicity of adenoviruses is closely linked to the amount of virus injected, the risk of immediate life threatening reactions is potentially much lower with replicating viruses.

The prototype tumor selective virus is a defective adenovirus lacking the EIB 55K gene (dl 1520/ONYX 015, Bischoff et al., 1996). In normal adenoviruses 55K inactivates p53, hence it should not be required in cells where p53 is mutant. In practice, many cells containing wild type p53 are killed by the virus (Heise et al., 1997). The present inventors have tested this in H1299 p53-null lung carcinoma cells containing wild type p53 under a tetracycline-regulated promoter and found that dl 1520 replicates as well in the presence as in the absence of wild type p53. Besides targeting p53, EIB 55K is required for selective viral RNA export (Shenk, 1996) and it is not immediately obvious how loss of p53 could substitute for this function. At present there is no convincing evidence that dl 1520 targets p53 defects (Goodrum 1997, Goodrum 1998, Hall 1998, Rothman 1998, Turnell 1999).

As with p53-expressing viruses, combination therapy with chemotherapy and dl 1520 gives better results both in vitro and in xenografts (Heise et al., 1997). In principle, the virus should undergo multiple rounds of replication until there are no tumor cells remaining and since each infected cell produces 10³ to 104 new virus particles, the amount of input virus should not be limiting. In practice, the required amount of dl 1520 virus injected is comparable for therapy with Ad-CMV-p53, a p53 supplementing virus. This means that the virus is not performing as expected for a replicating virus with the reasons for this again probably quite complex.

It is also possible to target early gene expression defects, as regulated by E2F, but this is complicated by the fact that as part of its life cycle the adenovirus already produces proteins (E1A and E4 orf 6/7) which target E2F. Since E1A and orf 6/7 are multifunctional proteins the effect of E1A and orf 6/7 mutations is complex and unpredictable.

In addition to E2F and p53, there are four transcription factors whose activity is known to increase in tumors. They are Tcf4, RBPJκ and Gli-1, representing the endpoints of the wnt, notch and hedgehog signal transduction pathways (Dahmane et al., 1997; Jarriault et al., 1995; van de Wetering et al., 1997) and HIF1alpha, which is stabilised by mutations in the Von Hippel Lindau tumor suppressor gene (Maxwell et al 1999). Mutations in APC or β-catenin are universal defects in colon cancer (Korinek et al., 1997; Morin et al., 1997); but they also occur at lower frequency in other tumors, such as melanoma (Rubinfeld et al., 1997). Such mutations lead to increased Tcf activity in affected cells. The hedgehog pathway is activated by mutations in the patched and smoothened proteins in basal cell cancer (Stone et al., 1996; Xie et al., 1998). Notch mutations occur in some leukaemias (Ellisen et al., 1991). Telomerase activation is one of the hallmarks of cancer (Hanahan D. and Weinberg R A. The hallmarks of cancer. Cell. 100, 57-70, 2000) and results from increased activity of the telomerase promoter, although the mechanism is unknown. According to Cong Y S et al (1999, HMG 8, 137-42) the elements responsible for promoter activity are contained within a region extending from 330 bp upstream of the ATG to the second exon of the gene and thus this sequence is a further suitable promoter sequence for use in the viral constructs and viruses of the invention.

Copending WO 00/56909, incorporated herein by reference, describes adenoviruses that replicate in response to activation of tumor specific transcription factors, particularly of the wnt signalling pathway. Wnt signalling is pathologically activated in virtually all colon tumors and this leads to transcription from promoters containing Tcf binding sites. The constitutive activation of the wnt pathway is caused by mutations in the APC, axin and β-catenin genes, thus inhibiting GSK-3′ phosphorylation of β-catenin and its subsequent degradation by the proteasome (34). Cytoplasmic β-catenin enters the nucleus, where it can associate with members of the Tcf/Lef family of transcription factors and activate transcription of wnt target genes, such as c-myc, cyclin D1, Tcf1 and matrilysin.

WO/00/56909 describes a viral construct in which Tcf binding sites are placed in the adenovirus E2 promoter, which regulates expression of the viral replication genes. Mutations elsewhere in the virus or cell cannot bypass the absolute requirement for E2 gene products in viral replication. In order to achieve tight regulation of E2 transcription, the adjacent E3 enhancer was also mutated. Tcf sites were also placed in the E1B promoter, although the level of regulation achieved did not affect viral replication in vitro. These “Tcf” viruses showed a 50 to 100-fold decrease in replication in non-permissive cell lines whereas their activity was comparable to wild type Ad5 in many colon cancer cell lines.

The present inventors have now found that some colon cell lines are only semi-permissive for the tumor specific viruses of WO 00/56909, making it desirable to alter the viral genome of these constructs to increase their breadth of effective activity to include these cells. Such broadening will also be calculable to increase efficacy against other tumors where the Tcf pathway is implicated, e.g. such as hepatocellular carcinoma and some breast, B cell, T cell, pancreatic, endometrial and ovarian cancers.

The present inventors have tested two different approaches to generate such viruses active in a broader range of colon cell lines: (i) insertion of tumor specific sites (e.g. Tcf sites as described above) in the E1A promoter region, and (ii) mutation of the p300 binding site in E1A. The wild type E1A enhancer contains two types of regulatory element, termed I and II, which overlap the packaging signal (See FIG. 1). In addition to elements I and II, there are transcription factor binding sites in the inverted terminal repeat (ITR) and close to the E1A TATA box.

The amino-terminus of E1A contains a region of E1A that binds p300, a histone acetylase which functions as a general transcription factor. E1A activates promoters that contain ATF sites. WO 00/56909 virus vMB13 retains the ATF site in the E3 promoter providing advantage in this respect. The problem is that if a promoter does not have an ATF site, E1A will repress it by binding p300. For example: E1A blocks p53-dependent transcription in a manner that requires the p300 binding site in E1A. Tcf repression by E1A is a possibility in some cell lines, so mutation of the E1A p300-binding site may be preferred for such treatment where Tcf is used for cellular targeting.

The present inventors see a difference between the previously disclosed vMB13 and vMB14 in HCT116 cells, where the only difference between the two viruses is in the ATF site in the E3 promoter. Thus mutation of the E1A p300-binding site in vMB14 might be advantageous. Alternatively, the difference could be due to direct activation of the ATF site because Xu L et al (2000, Genes Dev 14, 585-595) report that ATF/CREB sites can be activated by wnt signals, although the mechanism is unknown.

Thus in a first aspect of the present invention there is provided a viral DNA construct encoding for an adenovirus capable of replication in a human or animal tumor cell, and preferably causing death of such tumor cells, characterised in that it comprises one or more selected transcription factor binding sites operatively positioned together with the E1A open reading frame such as to promote expression of E1A proteins in the presence of said selected transcription factor, the level or activity of which factor being increased in a human or animal tumor cell relative to that of a normal human or animal cell of the same type, i.e. Lacking said transcription binding sites. Preferably the viral construct encodes for a virus that will cause death of the tumor cell directly, but in other embodiments it may encode a protein such as a vaccine, with the virus advantageously acting as adjuvant.

Preferably the viral DNA construct has a nucleic acid sequence corresponding to that of a wild type virus sequence characterised in that it has all or part of the wild type E1A transcription factor binding site replaced by the one or more selected transcription factor binding sites. More preferably the wild type E1A enhancer is deleted from its usual location or inactivated e.g. by mutation.

For the purposes of maintaining packaging capability of the construct the wild type packaging signal is preferably deleted from its wild type position (near the left hand inverted terminal repeat (ITR) in Ad5) and inserted elsewhere in the construct, in either orientation. Preferably the packaging signal is inserted adjacent the right hand terminal repeat, preferably within 600 bp of said ITR.

Preferably the E4 promoter contains the part of the E1A enhancer of the packaging signal flanked by Tcf and E4F sites.

Still more preferably one or more of the selected transcription factor binding sites are inserted into the right hand terminal repeat such as to provide sufficient symmetry to allow it to base pair to the left hand ITR during replication.

It will be realised from WO/00/56909 that the selected transcription factor binding sites are advantageously for a transcription factor whose activity or level is specifically increased by causal oncogenic mutations.

Preferably the nucleic acid sequence corresponds to that of the genome of an adenovirus with the selected transcription factor binding sites operatively positioned to control expression of the respective E1A genes. As with the viruses of WO 00/56909, the construct may advantageously have its nucleic acid sequence, other than the selected sites, corresponding to that of the genome of adenovirus Ad5, Ad40 or Ad41, or incorporates DNA encoding for fibre protein from Ad 5, Ad40 or Ad41, optionally with 1 to 30, more preferably 5 to 25, e.g. 15 to 25 lysines added to the end thereof.

Preferred constructs encode a functional viral RNA export capacity, e.g. they have an E1 region wherein the E1B 55K gene is functional and/or intact.

The preferred tumor specific transcription factor binding site used in place of wild type site is selected from Tcf-4, RBPJκ, Gli-1, HIF1alpha and telomerase promoter binding sites. Preferred transcription factor binding sites are selectively activated in tumor cells containing oncogenic APC and β-catenin mutations. e.g. the replacement sites are single or multiples of a Tcf-4 binding site sequence. e.g. comprising from 2 to 20 Tcf-4 binding site sequences at each replaced promoter site.

In addition to the essential substitution of control of E1A orf, one or more of the more selected transcription factor binding sites may also be operatively positioned together with one or more of the E1B, E2 and E3 open reading frame such as to promote expression of the E1B, E2 and E3 proteins in the presence of said selected transcription factor. Also preferably are mutations in one or more residues in the NF1, NFκB, AP1 and ATF regions of the E3 promoter. Preferably the E2 late promoter is also inactivated with silent mutations.

Viruses comprising or encoded by the DNA constructs described above are also provided.

In a further aspect is provided a viral DNA construct, or a virus, of the invention for use in therapy, particularly therapy of patients having neoplasms.

In a still further aspect is provided a viral DNA construct, or a virus, of the invention in the manufacture of a medicament for the treatment of neoplasms.

In a still further aspect of the present invention is provided a therapeutic composition comprising a viral construct, or a virus, of the invention together with a physiologically acceptable carrier. Particularly compositions are characterised in that they are sterile and pyrogen free with the exception of the presence of the viral construct or virus encoded thereby. For example the carrier may be a physiologically acceptable saline.

In a still further aspect is provided a method of manufacture of a viral DNA construct or a virus encoded thereby, as provided by the invention characterised in that it comprises transforming an adenovirus viral genome having one or more wild type transcription factor binding sites controlling transcription of E1A, and optionally E4 open reading frames, such as to replace one or more of these by tumor specific transcription factor binding sites. Preferred methods clone the viral genome by gap repair in a circular YAC/BAC in yeast. Preferably the genome is modified by gap repair into a mutant vector for modification of sequences near the ITRs or by two step gene replacement for modification of internal sequences. For example the modified genome may be transferred to a prokaryote for production of viral construct DNA. Preferably the genome is transferred to a mammalian cell for production of virus.

In a still further aspect of the present invention there is provided a method for treating a patient suffering from a neoplasm wherein a viral DNA construct or virus of the invention is caused to infect tissues of the patient, including or restricted to those of the neoplasm, and allowed to replicate such that neoplasm cells are caused to be killed.

To produce a tightly regulated tumor specific transcription factor driven virus, a mutant E1A promoter, such as a Tcf-E1A promoter, needs to be installed. To effect this the present inventors have substituted part of the left hand inverted terminal repeat (ITR) of the virus with tumor specific promoter, e.g. Tcf binding sites. More preferably the E1A enhancer is deleted from its wild type location, in part or in full, more preferably completely. Most preferably the packaging signal is relocated from its wild type site near the left hand ITR to another part of the viral genome where it is still effective to allow packaging of the virus. This is preferably relocated to adjacent the right hand ITR, more preferably to within 600 bp thereof. The packaging signal may be relocated in either orientation.

The tumor transcription factor specific promoter conveniently comprises one or more Tcf binding sites, more preferably two to ten, still more preferably three to five Tcf sites in tandem. Most preferably four Tcf binding sites replace a portion of the ITR, the E1A enhancer and the packaging signal on the left hand side while the packaging signal sequence is introduced adjacent the right hand ITR to permit proper encapsidation of viral DNA.

The right side substitutions are particularly desirable to maintain the symmetry of the terminal repeats, so a similar or identical number of tumor specific transcription factor binding sites are inserted in the right ITR as provided in the left ITR, such as to allow these sites to become base paired together during replication. It will be realised that these insertions are preferably substitutions as with the left side changes.

Tumour specific promoter-dependent transcription, e.g. with Tcf sites, is inhibited by E1A, so the inventors also investigated mutations in the E1A protein that would abolish this repression in transcription assays. Mutation of the p300 binding site in E1A partially relieved the repression, but in the context of the virus this mutation did not lead to increased transcription from the Tcf-E2 promoter and actually reduced the activity of the virus. Similar attenuation by mutation of the amino-terminus of E1A has been reported by the Onyx group. In contrast, it has now been surprisingly determined that the viruses containing only the transcription factor binding site changes in the E1A and E4 promoters (see for example vCF11 in the Examples herein) are selective for cells with active wnt signalling and active in most of the colon cancer cells studied.

Preferably the viruses of the invention also include tumor specific transcription factor binding sites in the promoter of the E2 open reading frame and more preferably also the promoter of the E3 open reading frame, as described in the copending patent WO 00/56909, which is incorporated herein by reference.

The Tcf sites in the preferred viruses of the present invention are adjacent to the TATA box in the Tcf-E1A promoter, but several hundred base pairs upstream of the E4 TATA box. To create an E1A promoter with the minimum possibility of interference from extraneous signals, all of the normal E1A regulatory elements were deleted from their wild type positions in a preferred construct and virus of the invention, vCF11.

This strategy contrasts with prior art approaches used to produce prostate, hepatocellular cancer and breast cancer targeting viruses, which retain the complete E1A enhancer but place exogenous promoters between it and the E1A start site. To remove the E1A enhancer in vCF11 it was necessary to transfer the viral packaging signal to the right ITR. In addition, approximately half of the right hand ITR was replaced by Tcf sites. This construction dictated the position of the Tcf sites relative to the E4 start site.

To optimise the Tcf-E4 promoter, it would be possible either to insert additional Tcf sites nearer the E4 start site or to delete the endogenous E4 control elements. The latter were retained in vCF11 because they confer repression of E4 transcription in normal cells. The mutant E4 promoter thus contains the part of the E1A enhancer contained in the packaging signal, which could activate the promoter, flanked by Tcf and E4F sites, which should repress the promoter in normal cells. The net result of these changes is reduced E4 transcription measured by luciferase assay, regardless of cell type.

Replication of the previous generation of viruses of WO 00/56909 is directed mainly at cells with activated wnt signalling by the Tcf sites in E2 promoter. The present invention viruses vCF22, 62 and 81, which have Tcf sites in multiple early promoters, are very selective but are relatively attenuated. The reduced activity in cytopathic effect assays seen with the viruses bearing mutations in all the early promoters might be due to deletion of element II in the E1A enhancer, which was previously reported to activate transcription of all early units in cis.

Comparison of different viruses shows that the Tcf-E1A promoter and Tcf-E2 promoters display the same hierarchy of activity in a panel of colon cell lines, but relative to the corresponding wild type promoters, the Tcf-E1A promoter is more active than the Tcf-E2 promoter. This probably explains why vCF11 is able to replicate better than vMB19 (see WO 00/56909) in Co115 cells.

To produce viruses that have substantially full spectrum activity using Tcf regulation of multiple early promoters is desirable to construct a Tcf-E2 promoter with much higher activity in the semi-permissive colon cells. Possible differences which could explain the reduced Tcf activity in some cell lines include increased expression of corepressors like groucho and CtBP, decreased expression of coactivators like p300 and CBP, pygopus, Bc19, acetylation or phosphorylation of Tcf4 preventing β-catenin binding or DNA binding, and increased activity of the ΔN-Tcf1 negative feedback loop.

Luciferase reporter assays show a systematic inhibition of Tcf-dependent transcription by E1A. Mutagenesis of E1A indicated that this effect was partly due to inhibition of p300 by E1A, consistent with reports that p300 is a coactivator for β-catenin. Coexpression of p300 together with E1A had the same effect on Tcf-dependent transcription as deletion of the p300 binding site in E1A, indicating that the remaining repression was unlikely to be due to inhibition of p300. The residual repressive effect of E1A could not be mapped to any known domain and merits further study. The negative results obtained with the ΔCR1 mutant are surprising because deletion of the CR1 p300-binding subdomain alone did partially restore Tcf-dependent transcription. This could conceivably be explained by an artefactual elevation of transcription of the renilla luciferase control by ΔCR1 E1A, but a more likely explanation is that another function of E1A is impaired by deletion of the entire CR1 domain.

The inhibition of Tcf-dependent transcription by E1A in the first generation viruses was greatest in the semi-permissive cell lines like Co115, resulting in very low luciferase activity because the starting level of Tcf activity was also lower in these cells. Hence, we expected to see a substantial effect of the Δ2-11 E1A mutation in the context of the viruses. In practice, the mutation produced no increase in expression from the Tcf promoters in colon cell lines and reduced the activity of the virus in cytopathic effect assays. The mutation had complex and inconsistent effects in burst assays: it appeared to reduce burst size in permissive cells when the E2 promoter was driven by E1A (i.e. wild type), but increase burst size in some non-permissive cells when the E2 promoter was driven by Tcf. A general explanation is that any gain in Tcf activity due to this E1A mutation was offset by a loss of other E1A activities. Since we only tested 12S E1A, it is possible that these functions map to the other E1A isoforms expressed during viral infection. In addition, there are some basal promoter activities regulated by E1A which may be abrogated by the Δ2-11 mutation.

The most mutant virus investigated, vCF62, lacks many of the transcriptional response elements through which E1A normally controls the virus (ATF sites in the E1A, E2, E3 and E4 promoters; E2F sites in the E2 promoter), and showed very large decreases in activity in semi-permissive cells in both burst and cytopathic effect assays.

Preferably the viral DNA construct is characterised in that it encodes a functional viral RNA export capacity. For adenovirus this is encoded in the E1 and E4 regions, particularly the E1B 55K and E4 orf 6 genes. Thus preferably the encoded virus is of wild type with respect to expression of these genes in tumor cells. Most preferably the E1B 55K and E4 orf 6 open reading frames are functional and/or intact where present in the corresponding wild type virus.

Preferred colon tumor specific adenoviruses are encoded by viral DNA constructs corresponding to the DNA sequence of Ad5 or one or more of the enteric adenoviruses Ad40 and Ad41 modified as described above. Ad40 and Ad41, which are available from ATCC, are selective for colon cells and one important difference to Ad5 is that there is an additional fibre protein. The fibre protein binds to the cell target host surface receptor, called the coxsackie-adeno receptor or CAR for Ad5. Colon cells have less CAR than lung cells which Ad5 is adapted to infect. Ad40 and Ad41 have two fibre proteins, with the possibility being that they may use two different receptors. The expected form of resistance to virus therapy is loss of the receptor, which obviously prevents infection. Genetic instability in tumors means this will happen at some reasonable frequency; about 1 in 100 million cells, a mutation rate of 1 in 10⁸. If you delete two receptors you multiply the probabilities; i.e. loss of both will occur in 1 in 10¹⁶ cells. A tumor contains between 10⁹ and 10¹² cells. Hence resistance is less likely to develop if a virus uses more than one receptor. One fibre protein in Ad40 and 41 uses CAR whilst the receptor used by the other is as yet unknown.

Advantageously the use of the constructs of the invention, particularly in the form of viruses encoded thereby, to treat neoplasms such as liver metastasis is relatively non-toxic compared to chemotherapy, providing good spread of virus within the liver aided by effective replication.

Preferred tumor specific transcription factor binding sites that are used in place of wild type sites are those described above as Tcf-4, HIF1alpha, RBPJκ and Gli-1 sites, and a fragment of the telomerase promoter conferring tumor-specific transcription.

A most preferred transcription factor binding site is that which binds Tcf-4, such as described by Vogelstein et al in U.S. Pat. No. 5,851,775 and is responsive to the heterodimeric β-catenin/Tcf-4 transcription factor. As such the transcription factor binding site increases transcription of genes in response to increased β-catenin levels caused by APC or β-catenin mutations. The telomerase promoter is described by Wu K J. et al (1999, Nat Genet 21, 220-4) and Cong YS. et al (1999 HumMol Genet 8, 137-42). A further preferred binding site is that of HIF1alpha, as described by Maxwell P H. et al, (1999 Nature 399, 271-5). One may use a HIF1alpha-regulated virus to target the hypoxic regions of tumors, involving no mutation of the pathway as this is the normal physiological response to hypoxia, or the same virus may be used to target cells with VHL mutations either in the familial VHL cancer syndrome, or in sporadic renal cell carcinomas, which also have VHL mutations. A retrovirus using the HIF promoter to target hypoxia in ischemia has already been described by Boast K. et al (1999 Hum Gene Ther 10, 2197-208).

Particularly the inventors have now provided viral DNA constructs. and viruses encoded thereby, which contain the Tcf transcription factor binding sites referred to above in operational relationship with the E1A, and optionally E4, open reading frames described above, particularly in place of wild type transcription factor binding sites in their promoters and shown that these are selective for tumor cells containing oncogenic APC and β-catenin mutations. Tcf-4 and its heterodimer bind to a site designated Tcf herein. Preferred such replacement sites are single or multiples of the Tcf binding sequence, e.g. containing 2 to 20, more preferably 2 to 6, most conveniently, 2, 3 or 4 Tcf sites.

Particular Tcf sites are of consensus sequence (A/T)(A/T)CAA(A/T)GG, see Roose, J., and Clevers, H. (1999 Biochim Biophys Acta 1424, M23-37), but are more preferably as shown in the examples herein.

A preferred group of viral constructs and viruses of the invention are those having the further selected transcription factor binding site in a function relationship with the E2 orfs and more preferably also with the E3 orfs. Preferably the VIII region containing the E3 promoter is characterised in that it has mutations to one or more residues in the NF1, NFκB, API and/or ATF regions of the E3 promoter, more preferably those mutations which reduce E2 gene transcription caused by E3 promoter activity. The present inventors have particularly provided silent mutations, these being such as not to alter the predicted protein sequence of any viral protein but which alter the activity of key viral promoters.

NFκB is strongly induced in regenerating liver cells, ie. hepatocytes (see Brenner et al J. Clin. Invest. 101 p802-811). Liver regeneration to fill the space vacated by the tumor is likely to occur following successful treatment of metastases. In addition, if one wishes to treat hepatoma, which arise on a background of dividing normal liver cells, then destroying the NFκB site is potentially advantageous.

E1A normally activates the E2 promoter through the ATF site. In the absence of such targeting E1A represses promoters, eg. by chelating p300/CBP. When the ATF site is deleted in a mutant E2 promoter, E1A produced by the virus should reduce general leakiness of the mutant E2 promoter in all cell types. The E3 promoter is back-to-back with the E2 promoter and the distinction between them is defined but functionally arbitrary. Hence further reduction of the activity of the mutant E2 promoter is possible by modifying or deleting transcription factor binding sites in the E3-promoter. Since the E3 promoter lies in coding sequence it cannot just be deleted. Instead the inventors have provided up to 16 silent substitutions changing critical residues in known NFI, NFκB, API and ATF sites (Hurst and Jones, 1987, Genes Dev 1, 1132-46, incorporated herein by reference).

Further viral constructs of the present invention may be provided by modifying the E2-late promoter of adenoviruses. The E2-early promoter controls transcription of DNA polymerase (pol), DNA binding protein (DBP) and preterminal protein (pTP). By mutating the E2 late promoter it is possible to have a similar effect, ie. at least in part, to the E1B deletion because E1B deletion reduces export of DBP RNA expressed from the E2 late promoter. DBP is required stoichiometrically for DNA replication, so reducing DBP production in normal cells is desirable. Since the E2 late promoter lies in 100 k protein coding sequence it cannot just be deleted. Instead the inventors have determined that it can inactivated with silent mutations changing critical residues in known transcription factor binding sites.

Particular transcription factor binding sites in the E2 late promoter were identified by DNase I footprinting (marked I-IV in FIG. 4 herein; Goding et al, 1987, NAR 15, 7761-7780). The most important is a CCAAT box lying in footprint II. Mutation of this CCAAT box reduces E2 late promoter activity 100-fold in CAT assays (Bhat et al, 1987,EMBO J, 6,2045-2052). One such mutation changes the marked CCAAT box sequence GAC CAA TCC to GAT CAG TCC. (see FIG. 4 below). This is designed to abolish binding of CCAAT box binding factors without changing the 100 k protein sequence. Additional silent mutations in the other footprints can be used to reduce activity further

An further preferred or additional mutation possible is to regulate expression of E1B transcription by mutating the E1B promoter. This has been shown to reduce virus replication using a virus in which a prostate-specific promoter was used to regulate E1B transcription (Yu, D. C., et al 1999 Cancer Research 59, 1498-504). A further advantage of regulating E1B 55K expression in a tumor-specific manner would be that the risk of inflammatory damage to normal tissue would be reduced (Ginsberg, H. S., et al 199 PNAS 96, 10409-11). The inventors have produced viruses with Tcf sites replacing the E1B promoter Spl site to test this proposition.

In contrast with, for example, the Calydon viruses, the design of the present inventors viruses means that, despite retaining a full complement of adenoviral genes, spare packaging capacity is available, which can be used to express conditional toxins, such as the prodrug-activating enzyme HSV thymidine kinase (tk), nitroreductase (eg. from E. coli-see Sequence listing), cytosine deaminase (eg from yeast-m see Sequence listing). This could be expressed for example from the E3 promoter, whose activity is regulated in some of the viruses, to provide an additional level of tumor targeting. Alternatively, it could be expressed from a constitutive promoter to act as a safety feature, since ganciclovir would then be able to kill the virus. Constitutive tk expression in an E1B-deficient virus also increases the tumor killing effect, albeit at the expense of replication (Wildner, O., et al 1999 Gene Therapy 6, 57-62). An alternative prodrug-activating enzyme to express would be cytosine deaminase (Crystal, R. G., et al 1997 Hum Gene Ther 8, 985-1001), which converts 5FC to 5FU. This has advantage because 5FU is one of the few drugs active on liver metastases, the intended therapeutic target, but produces biliary sclerosis in some patients.

In a preferred virus the ‘suicide gene’ eg sequence encoding the toxin, is expressed from a position between the fiber and the E4 region. This gene is preferably and expressed late either with an IRES or by differencial splicing, that is, in a replication-dependant manner. Such aspect is novel and inventive in its own right and forms an independent invention.

Having produced a virus with one or more levels of regulation to prevent or terminate replication in normal cells, it is further preferred and advantageous to improve the efficiency of infection at the level of receptor binding. The normal cellular receptor for adenovirus, CAR, is poorly expressed on some colon tumor cells. Addition of a number of lysine residues, eg 1 to 25, more preferably about 5 to 20, to the end of the adeno fibre protein (the natural CAR ligand) allows the virus to use heparin sulphate glycoproteins as receptor, resulting in more efficient infection of a much wider range of cells. This has been shown to increase the cytopathic effect and xenograft cure rate of E1B-deficient viruses (Shinoura, H., et al 1999 Cancer Res 59, 3411-3416 incorporated herein by reference). Fibre mutations that alter NGR, PRP or RGD targeting may also be expolited, eithre increasing or decreasing such effect depending upon the need to increase or decrease infectivity toward given cell types.

An alternative strategy is to incorporate the cDNA encoding for Ad40 and/or Ad41 fibres, or other efficaceous fibre type such as Ad3 and Ad35 into the construct of the invention as described above. The EMBL and Genbank databases list such sequences and they are further described in Kidd et al Virology (1989) 172(1), 134-144; Pieniazek et al Nucleic Acids Res. (1989) Nov 25;17-20, 9474; Davison et al J. Mol. Biol (1993) 234(4) 1308-16; Kidd et al Virology (1990) 179(1) pl39-150; all of which are incorporated herein by reference.

In a second aspect of the invention there is provided the viral DNA construct of the invention, particularly in the form of a virus encoded thereby, for use in therapy, particularly in therapy of patients having neoplasms, eg. malignant tumors, particularly colorectal tumors and most particularly colorectal metastases. Most preferably the therapy is for liver tumors that are metastases of colorectal tumors.

In a third aspect there is provided the use of a viral DNA construct of the invention, particularly in the form of a virus encoded thereby, in the manufacture of a medicament for the treatment of neoplasms, eg. malignant tumors, particularly colorectal tumors and most particularly colorectal metastases. Most preferably the treatment is for liver tumors that are metastases of colorectal tumors.

In a fourth aspect of the invention there are provided compositions comprising the viral DNA construct of the invention, particularly in the form of a virus encoded thereby, together with a physiologically acceptable carrier. Such carrier is typically sterile and pyrogen free and thus the composition is sterile and pyrogen free with the exception of the presence of the viral construct component or its encoded virus. Typically the carrier will be a physiologically acceptable saline.

In a fifth aspect of the invention there is provided a method of manufacture of the viral DNA construct of the invention, particularly in the form of a virus encoded thereby comprising transforming a viral genomic DNA, particularly of an adenovirus, having wild type E1A transcription factor binding sites, particularly as defined for the first aspect, such as to operationally replace these sites by tumor specific transcription factor binding sites, particularly replacing them by Tcf transcription factor binding sites. Operational replacement may involve partial or complete deletion of the wild type site. Preferably the transformation inserts two or more, more preferably 3 or 4, Tcf-4 transcription factor binding sites. More preferably the transformation introduces additional mutations to one or more residues in the NF1, NFκB, API and/or ATF binding sites in the E3 promoter region of the viral genome. Such mutations should preferably eliminate interference with E2 activity by E3 and reduce expression of E2 promoter-driven genes in normal cells and non-colon cells. Reciprocally, it preferably replaces normal regulation of E3 with regulation by Tcf bound to the nearby E2 promoter.

Traditional methods for modifying adenovirus require in vivo reconstitution of the viral genome by homologous recombination, followed by multiple rounds of plaque purification. The reason for this is the difficulty of manipulating the 36 kb adenovirus genome using traditional cloning techniques. Newer approaches have been developed which circumvent this problem, particularly for E1-replacement vectors. The Transgene and Vogelstein groups use gap repair in bacteria to modify the virus (Chartier et al., 1996; He et al., 1998). This requires the construction of large vectors which are specific for each region to be modified. Since these vectors are available for E1-replacement, these approaches are very attractive for construction of simple adenoviral expression vectors. Ketner developed a yeast-based system where the adenoviral genome is cloned in a YAC and modified by two step gene replacement (Ketner et al., 1994). The advantage of the YAC approach is that only very small pieces of viral DNA need ever be manipulated using conventional recombinant DNA techniques. Conveniently, a few hundred base pairs on either side of the region to be modified are provided and on one side there should be a unique restriction site, but since the plasmid is very small this is not a problem. The disadvantage of the Ketner approach is that the yield of YAC DNA is low.

The present inventors have combined the bacterial and yeast approaches which may contain mutant viral sequences. Specifically, they clone the viral genome by gap repair in a circular YAC/BAC in yeast, modify it by two step gene replacement, then transfer it to bacteria for production of large amounts of viral genomic DNA. The latter step is useful because it permits direct sequencing of the modified genome before it is converted into virus, and the efficiency of virus production is high because large amounts of genomic DNA are available. They use a BAC origin to avoid rearrangement of the viral genome in bacteria. Although this approach has more steps, it combines all of the advantages and none of the disadvantages of the pure bacterial or yeast techniques.

Although it can be used to make E1-replacement viruses, and the inventors have constructed YAC/BACs allowing cycloheximide selection of desired recombinants in the yeast excision step to simplify this task, the main strength of the approach is that it allows introduction of mutations at will throughout the viral genome. Further details of the YAC/BAC are provided by the inventors as their contribution to Gagnebin et al (1999) Gene Therapy 6, 1742-1750) which is incorporated herein by reference.:Sequential modification at multiple different sites is also possible without having to handle large DNA intermediates in vitro.

The adenovirus strain to be mutated using the method of the invention is preferably a wild type adenovirus. Conveniently adenovirus 5 (Ad 5) is used, as is available from ATCC as VR5. The viral genome is preferably completely wild type outside the regions modified by the method, but may be used to deliver tumor specific toxic heterologous genes, eg. p53 or genes encoding prodrug-activating enzymes such as thymidine kinase which allows cell destruction by ganciclovir. However, the method is also conveniently applied using viral genomic DNA from adenovirus types with improved tissue tropisms (eg. Ad40 and Ad41).

In a sixth aspect of the present invention there is provided a method for treating a patient suffering from neoplasms wherein a viral DNA construct of the invention, particularly in the form of a virus encoded thereby, is caused to infect tissues of the patient, including or restricted to those of the neoplasm, and allowed to replicate such that neoplasm cells are caused to be killed.

The present invention further attempts to improve current intra-arterial hepatic chemotherapy by prior administration of a colon-targeting replicating adenovirus. DNA damaging and antimetabolic chemotherapy is known to sensitise tumor cells to another replicating adenovirus in animal models (Heise et al., 1997). For example, during the first cycle the present recombinant adenovirus can be administered alone, in order to determine toxicity and safety. For the second and subsequent cycles recombinant adenovirus can be administered with concomitant chemotherapy. Safety and efficacy is preferably evaluated and then compared to the first cycle response, the patient acting as his or her own control.

Route of administration may vary according to the patients needs and may be by any of the routes described for similar viruses such as described in U.S. Pat. No. 5,698,443 column 6, incorporated herein by reference. Suitable doses for replicating viruses of the invention are in theory capable of being very low. For example they may be of the order of from 10² to 10¹³, more preferably 10⁴ to 10¹¹, with multiplicities of infection generally in the range 0.001 to 100.

For treatment a hepatic artery catheter, eg a port-a-cath, is preferably implanted. This procedure is well established, and hepatic catheters are regularly placed for local hepatic chemotherapy for ocular melanoma and colon cancer patients.

A baseline biopsy may be taken during surgery.

A typical therapy regime might comprise the following:

Cycle 1: adenovirus construct administration diluted in 100 ml saline through the hepatic artery catheter, on days 1, 2 and 3.

Cycle 2 (day 29): adenovirus construct administration on days 1, 2, and 3 with concomitant administration of FUDR 0.3 mg/kg/d as continuous infusion for 14 days, via a standard portable infusion pump (e.g. Pharmacia or Melody), repeated every 4 weeks.

Toxicity of viral agent, and thus suitable dose, may be determined by Standard phase I dose escalation of the viral inoculum in a cohort of three patients. If grade III/IV toxicity occurs in one patient, enrolment is continued at the current dose level for a total of six patients. Grade III/V toxicity in≧50% of the patients determines dose limiting toxicity (DLT), and the dose level below is considered the maximally tolerated dose (MTD) and may be further explored in phase II trials.

It will be realised that GMP grade virus is used where regulatory approval is required.

It will be realised by those skilled in the art that the administration of therapeutic adenoviruses may be accompanied by inflammation and or other adverse immunological event which can be associated with eg. cytokine release. Some viruses according to the invention may also provoke this, particularly if E1B activity is not attenuated. It will further be realised that such viruses may have advantageous anti-tumor activity over at least some of those lacking this adverse effect. In this event it is appropriate that an immuno-suppressive, anti-inflammatory or otherwise anti-cytokine medication is administered in conjunction with the virus, eg, pre-, post- or during viral adminstration. Typical of such medicaments are steroids, eg, prednisolone or dexamethasone, or anti-TNF agents such as anti-TNF antibodies or soluble TNF receptor, with suitable dosage regimes being similar to those used in autoimmune therapies. For example, see doses of steroid given for treating rheumatoid arthritis (see WO93/07899) or multiple sclerosis (WO93/10817), both of which in so far as they have US equivalent applications are incorporated herein by reference.

In conclusion, we have shown that adenovirus replication can be regulated by insertion of Tcf sites into the E1A or E2 promoters. Mutation of the p300 binding site in E1A did not increase transcription from Tcf promoters in the context of the virus. Since the Δ2-11 mutation consistently reduced virus activity in cytopathic effect assays, it would be better to retain the p300 2-11 domain in therapeutic viruses.

To achieve strong activation of viral E2 transcription in cell lines with only weak Tcf activity will require the insertion of sites for synergistically acting transcription factors or modification of the basal promoter.

The present invention will now be described by way of illustration only by reference to the following non-limiting Examples, Methods, Sequences and Figures. Further embodiments falling within the scope of the claims will occur to those skilled in the art in the light of these.

TABLE 1
Structure of the adenoviruses used in this study

| virus | mutant | Promoters | | | | | ORF |
|---|---|---|---|---|---|---|---|
| name | regions^{a} | E1A | E1B | E2 | E3 | E4 | E1A |
| vCF11 | A4 | Tcf^{b} | wt | wt | wt | mut^{c} | wt |
| vCF42 | AΔ4 | Tcf | wt | wt | wt | mut | Δp300^{d} |
| vMB31 | B23′ | wt | Tcf | Tcf | mut + A^{e} | wt | wt |
| vCF22 | AB23′4 | Tcf | Tcf | Tcf | mut + A | mut | wt |
| vKH1 | AΔ4 | Tcf | Tcf | wt | wt | mut | wt |
| vMB19 | B23 | wt | Tcf | Tcf | mut − A^{f} | wt | wt |
| vCF81 | ΔB23 | wt | Tcf | Tcf | mut − A | wt | Δp300 |
| vCF62 | AΔB234 | Tcf | Tcf | Tcf | mut − A | mut | Δp300 |
| VCaK1 | ABFIS4 | Tcf | Tcf | wt | wt | mut | wt^{g} |

^{a}Abbreviations used in FIG. 3.
^{b}Replacement of endogenous promoters by four Tcf binding sites.
^{c}Insertion of three Tcf binding sites and the packaging signal upstream of the endogenous promoter.
^{d}Deletion of amino acids 2-11 in E1A.
^{e}Mutation of the NF1, NFκB, and AP1 sites in the E3 promoter.
^{f}Mutation of the NF1, NFκB, AP1, and ATF sites in the E3 promoter.
^{g}Mutations of HSPG and CAR binding domain of fibre + insertion of RGD4c peptide in fibre H1 loop in CaK1 fibre + EMCV IRES driving translation of yeast cytosine deaminase from the late major transcript.

### FIGURES

FIG. 1.

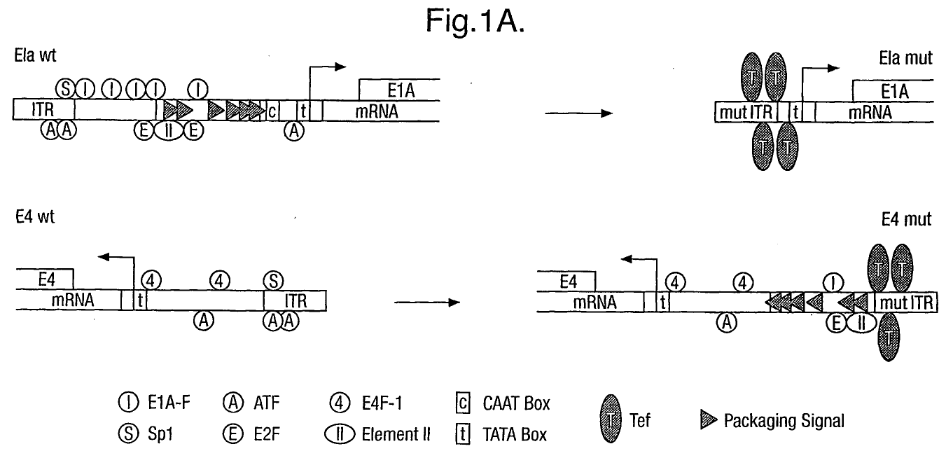

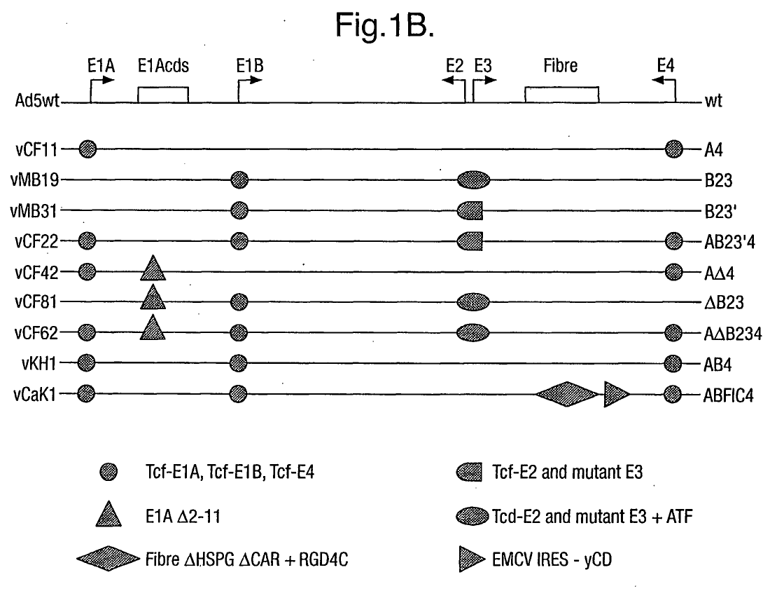

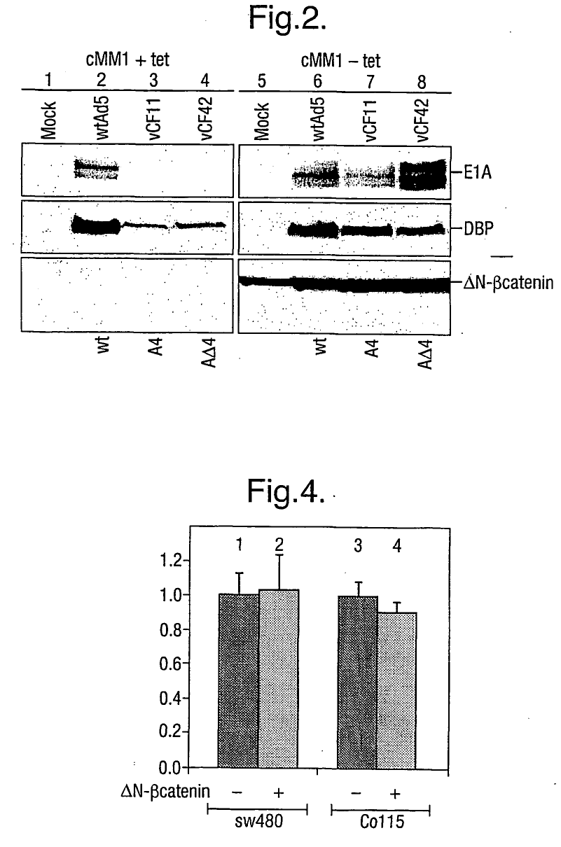

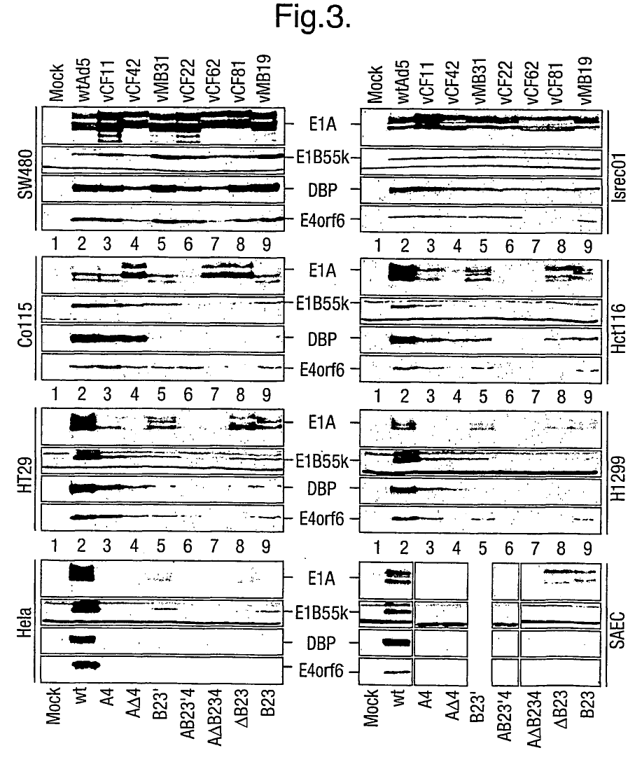

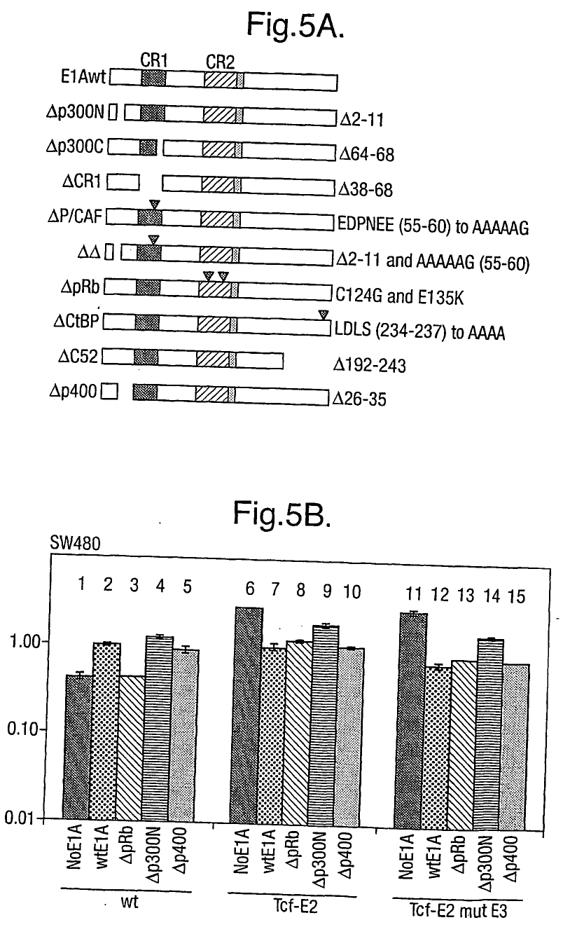

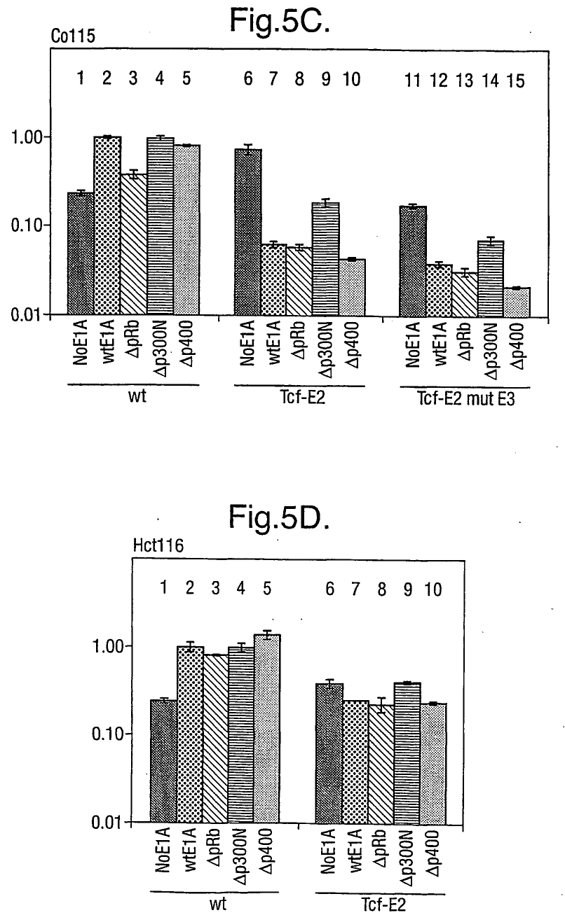

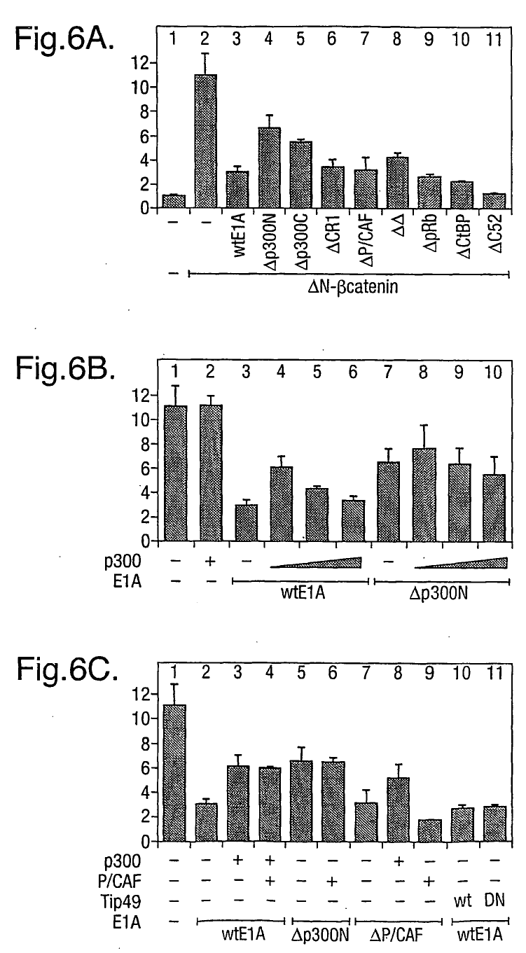

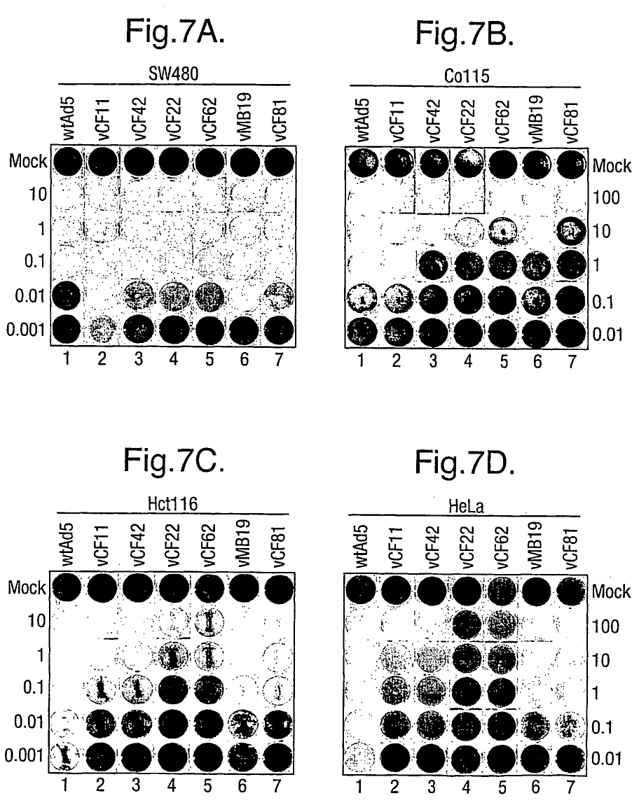

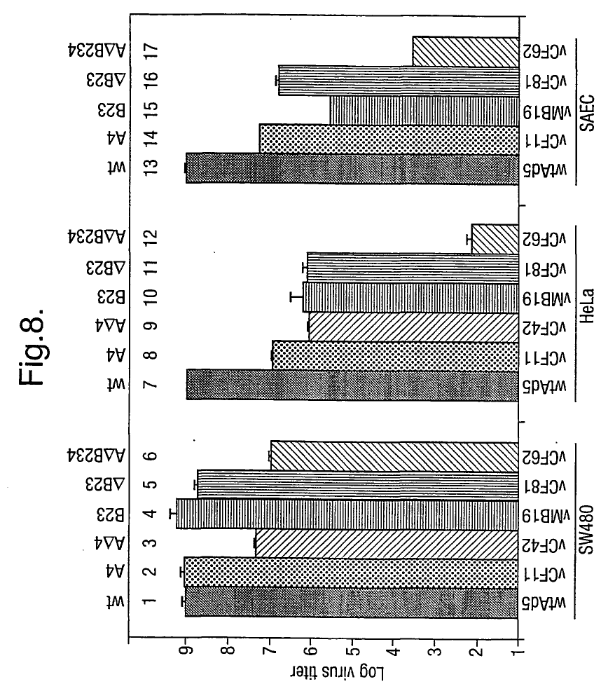

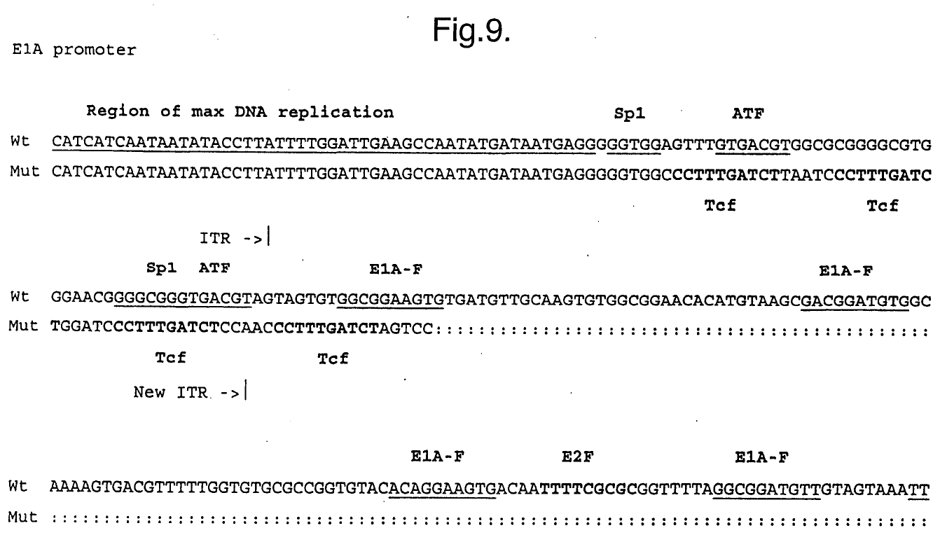

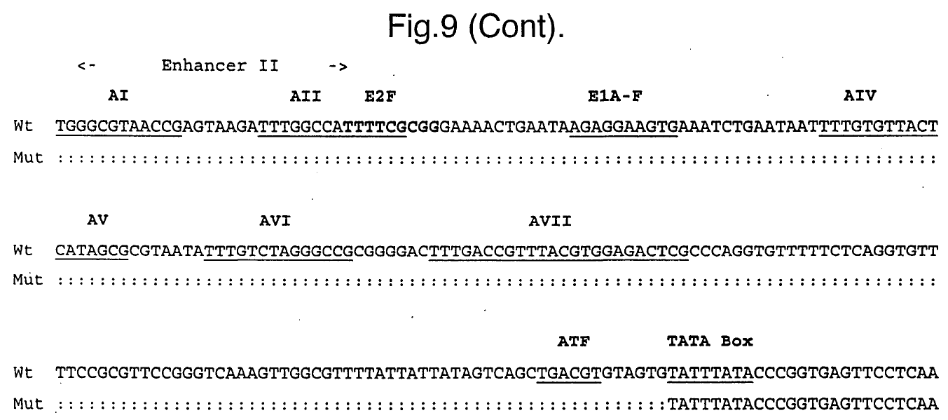

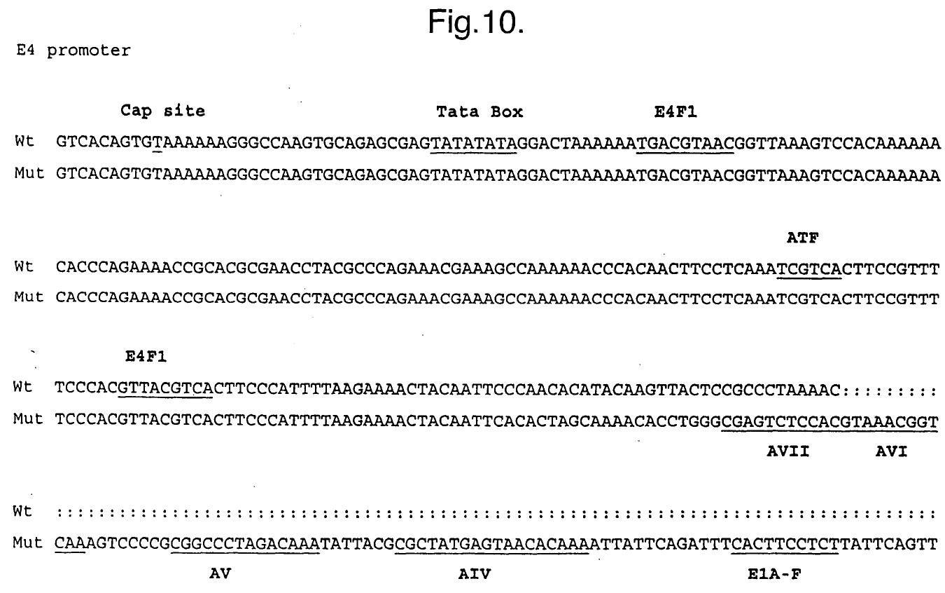

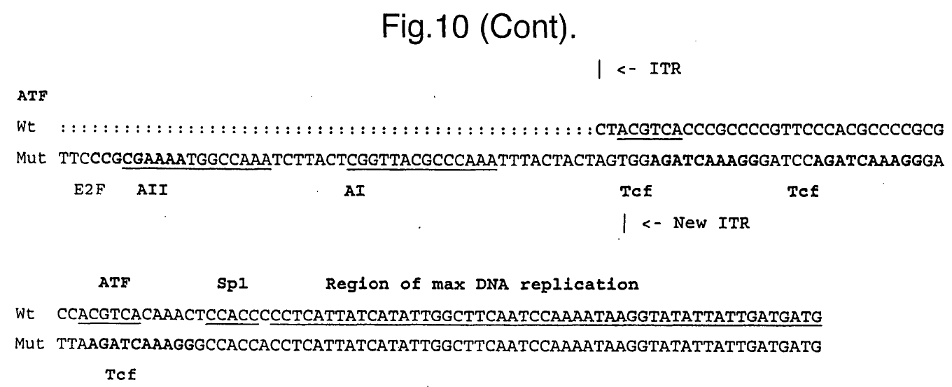

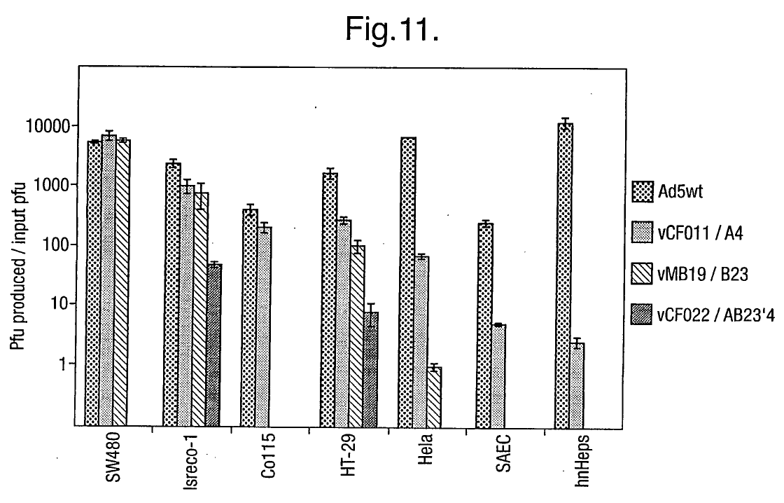

(A) Schematic diagram showing the mutagenesis of the E1A promoter (upper part) and E4 promoter (lower part). Both regions are shown from the ITRs to the beginning of the first open reading frame. The dark triangles represent the A motifs in the packaging signal.

(B) Schematic diagram showing mutant regions in the viruses used in this study (see table 1 for details). To facilitate interpretation of the figures, the viruses are given clone names (vCFs and vMBs) and a codename summarising their structure: A, B, 2, 4=Tcf sites in the E1A, E1B, E2, and E4 promoters, respectively. 3=silent mutations in the NF1, NFκB, AP1, and ATF sites in the E3 promoter.3′=as 3, but without the ATF site mutation. Δ=deletion of amino acids 2-11 in E1A that abolishes p300 binding. F=mutations in the fibre that abolish HSPG and CAR binding together with insertion of an RGD4C peptide in the H1 loop. I=EMCV IRES. C=Yeast cytosine deaminase.

FIG. 2: Western blot of cMM1 cells probed for E1A and DBP 24 hours after infection with wild type Ad5 and Tcf-viruses. Tetracycline withdrawal leads to expression of ΔN-β-catenin (lanes 6-8). The Tcf-E1A promoter responds to activation of wnt signalling (lane 7).

FIG. 3. Western blot for E1A, E1B55k, DBP and E4orf6 24 hours after infection of different cell lines with wild-type Ad5 and Tcf viruses. SW480 and Isrec01 are permissive colon cancer cell lines. Co115, Hct116 and HT29 are semi-permissive colon cancer cell lines. H1299, HeLa and SAEC are non-permissive cell lines in which the wnt pathway is inactive. (The SAEC blot is derived from two separate experiments giving similar wild-type Ad5 activity. vMB31 was not tested on SAEC)

FIG. 4. Bar chart of results of luciferase assays in SW480 and Co115 using a Tcf-E2 reporter; shows β-catenin is not limiting in SW480 and Co115 colon cancer cell lines.

FIG. 5. E1A inhibits Tcf-dependent-transcription. (A) Schematic diagram of the E1A12S mutants. (B-D) Luciferase assays with a wild-type E2 reporter and Tcf-E2 reporters. The “Tcf-E2 mut E3” reporter contains inactivating mutations in the E3 enhancer (9). Cells were transfected with luciferase reporters and plasmids expressing E1A mutants (shown in A). (B) SW480, (C) Co115, (D) Hct116.

FIG. 6. Luciferase assays in the lung cancer cell line H1299 showing inhibition of Tcf-dependent transcription by mutant forms of E1A. (A) Cotransfection of a Tcf-E1A reporter with various E1A mutants and ΔN-β-catenin. (B) Cotransfection of increasing amounts of p300 plasmid (0.5, 1, or 2 μg) lead to a decrease in Tcf-dependent transcription. (C) Effect of p300, P/CAF and Tip49 on Tcf-dependent transcription in the presence of wild-type and mutant forms of E1A. The values represent the fold activation versus the E1A wild-type reporter in the absence of E1A and ΔN-β-catenin.

FIG. 7. Cytopathic effect assays in different cell lines infected with 10-fold dilutions of wild type AdS and Tcf viruses. (A) SW480 cells were infected at a starting multiplicity of 10 pfu/cell and stained 6 days after infection. (B) Co115 and (C) Hct116 were infected at a starting multiplicity of 100 pfu/cell and stained 7 days after infection. (D) HeLa were infected at a starting multiplicity of 100 pfu/cell and stained 8 days after infection.

FIG. 8. Viral burst assays on permissive and non-permissive cell lines. SW480, Hela and SAEC cells were infected with 300 viral particles/cell and lysed 48 hours after infection. The titre of viral particles present in the lysate was measured by plaque assay on SW480. Values were normalised to the wild type AdS titre on each cell line. *vCF42 was not tested on SAEC.

FIG. 9. Comparison of sequences of wild type AdS E1A promoter and Tcf mutation E1A promoter of the present invention.

FIG. 10. Comparison of sequences of wild type AD5 E4 promoter and Tcf mutation E4 promoter of the present invention.

FIG. 11. Burst Assay results shown as histogram for a number of cell lines infected by Ad5 wt and three viruses of the invention.

1. A viral DNA construct encoding for an adenovirus capable of replication in a human or animal tumor cell characterised in that it comprises one or more selected transcription factor binding sites operatively positioned together with the E1A open reading frame such as to promote expression of E1A proteins in the presence of said selected transcription factor, the level or activity of which factor being increased in a human or animal tumor cell relative to that of a normal human or animal cell of the same type.

2. A viral DNA construct as claimed in claim 1 having a nucleic acid sequence corresponding to that of a wild type virus sequence characterised in that it has all or part of the wild type E1A transcription factor binding site replaced by the one or more selected transcription factor binding sites.

3. A viral DNA construct as claimed in claim 1 or 2 characteried in that the wild type E1A enhancer is deleted.

4. A viral DNA construct as claimed in any one of claims 1 to 3 characterised in that the wild type packaging signal is deleted from its wild type site adjacent the left hand inverted terminal repeat (ITR) and inserted elsewhere in the construct, in either forward or backward orientation.

5. A viral DNA construct as claimed in any one of claims 1 to 4 characterised in that the packaging signal is inserted adjacent, preferably within 600 bp, the right hand terminal repeat.

6. A viral DNA construct characterised in that one or more of the selected transcription factor binding sites are inserted into the right hand terminal repeat such as to provide sufficient symmetry to allow it to base pair to the left hand ITR during replication.

7. A viral DNA construct as claimed in any one of the preceding claims characterised in that the selected transcription factor binding sites are for a transcription factor whose activity or level is specifically increased by causal oncogenic mutations.

8. A construct as claimed in claim 7 characterised in that its nucleic acid sequence corresponds to that of the genome of an adenovirus with the selected transcription factor binding sites operatively positioned to control expression of the respective genes.

9. A construct as claimed in any one of the preceding claims characterised in that its nucleic acid sequence, other than the selected sites, corresponds to that of the genome of adenovirus Ad5, Ad40 or Ad41, or incorporates DNA encoding for fibre protein from Ad 5, Ad40 or Ad41, optionally with 15 to 25 lysines added to the end thereof.

10. A construct as claimed in any one of the preceding claims characterised in that it encodes a functional viral RNA export capacity.

11. A construct as claimed in any one of the preceding claims having an E1 region wherein the E1B 55K gene is functional and/or intact.

12. A construct as claimed in any one of the preceding claims characterised in that the tumor specific transcription factor binding site used in place of wild type site is selected from Tcf-4, RBPJκ, Gli-1, HIF1 alpha and telomerase promoter binding sites.

13. A construct as claimed in any one of the preceding claims characterised in that the substituting transcription factor binding site is selectively activated in tumor cells containing oncogenic APC and β-catenin mutations.

14. A construct as claimed in any one of the preceding claims characterised in that the replacement sites are single or multiples of a Tcf-4 binding site sequence.

15. A construct as claimed in claim 14 characterised in that it comprises from 2 to 20 Tcf-4 binding site sequences at each replaced promoter site.

16. A construct as claimed in any one of the preceding claims characterised in that it also has one or more of the more selected transcription factor binding sites operatively positioned together with one or more of the E1B, E2 and E3 open reading frame such as to promote expression of one or more E1B, E2 and E3 proteins in the presence of said selected transcription factor.

17. A construct as claimed in any one of the preceding claims characterised in that its sequence corresponds to that of an adenovirus genome having mutations in one or more residues in the NF1, NFκB, AP1 and ATF regions of the E3 promoter.

18. A construct as claimed in any one of the preceding claims characterised in that its sequence corresponds to that of an adenovirus genome wherein the E2 late promoter has been inactivated with silent mutations.

19. A construct as claimed in any one of the preceding claims characterised in that the E4 promoter contains the part of the E1A enhancer of the packaging signal flanked by Tcf and E4F sites.

20. A virus comprising or encoded by a DNA construct as claimed in any one of claims 1 to 19.

21. A viral DNA construct, or a virus, as claimed in any one of claims 1 to 19 for use in therapy.

22. A viral DNA construct, or a virus, as claimed in claims 20 or claim 21 characterised in that the therapy is of patients having neoplasms.

23. A viral construct or virus as claimed in any one of claims 1 to 22 characterised in that it is capable of causing death of the tumor cell.

24. Use of a viral construct, or a virus, as claimed in any one of claims 1 to 23 in the manufacture of a medicament for the treatment of neoplasms.

25. A composition comprising a viral construct, or a virus, as claimed in any one of claims 1 to 23 together with a physiologically acceptable carrier.

26. A composition as claimed in claim 25 characterised in that it is sterile and pyrogen free with the exception of the presence of the viral construct or virus encoded thereby.

27. A composition as claimed in claim 25 or 26 characterised in that the carrier is a physiologically acceptable saline.

28. A method of manufacture of a viral DNA construct or a virus encoded thereby as claimed in any one of claims 1 to 23 characterised in that it comprises transforming a viral genome having one or more wild type transcription factor binding sites controlling transcription of E1A, and optionally E4 open reading frames, such as to replace one or more of these by tumor specific transcription factor binding sites.

29. A method as claimed in claim 28 characterised in that the viral genome is cloned by gap repair in a circular YAC/BAC in yeast.

30. A method as claimed in claim 28 or 29 characterised in that the genome is modified by two step gene replacement.

31. A method as claimed in claim 28, 29 or 30 characterised in that the modified genome is transferred to a prokaryote for production of viral construct DNA.

32. A method of manufacture of a virus characterised in that viral construct DNA produced by a method as claimed in any one of claims 28 to 31, is transferred to a mammalian cell for production of virus.

33. A method for treating a patient in need of therapy for a neoplasm wherein a viral DNA construct or virus as claimed in any one of claims 1 to 23 is caused to infect tissues of the patient, including or restricted to those of the neoplasm, and allowed to replicate such that neoplasm cells are caused to be killed.

34. A method as claimed in claim 33 characterised in that the patient is in need of therapy for a colon cell derived tumor.

35. A method as claimed in claim 34 charactersied in that the colon cell-derived tumor is a metastasis located in the liver of the patient.
